(19) United States
(12) Patent Application Publication
(10) Pub. No.: US 2024/0140936 A1
(43) Pub. Date: May 2, 2024

(54) METHOD FOR THE PREPARATION OF PYRAZOLE DERIVATIVES AS MODULATORS OF CFTR PROTEIN

(71) Applicants: FONDAZIONE ISTITUTO ITALIANO DI TECNOLOGIA, Genova (IT); FONDAZIONE PER LA RICERCA SULLA FIBROSI CISTICA ETS, Verona (IT)

(72) Inventors: Fabio Bertozzi, Genova (IT); Tiziano Bandiera, Genova (IT); Francesco Berti, Genova (IT); Alejandra Rodriguez Gimeno, Genova (IT); Federico Sorano, Verona (IT); Filippo Romoli, Genova (IT); Francesco Calogero, Genova (IT)

(21) Appl. No.: 18/276,519

(22) PCT Filed: Feb. 18, 2022
(86) PCT No.: PCT/IB2022/051473

§ 371 Date: Aug. 9, 2023

(30) Foreign Application Priority Data
Feb. 18, 2021 (IT) 102021000003794

(51) Int. Cl. C07D 405/12

(52) U.S. Cl. CPC C07D 405/12

(57) ABSTRACT

The present invention relates to a method for the enantioselective preparation of a compound of formula (I).

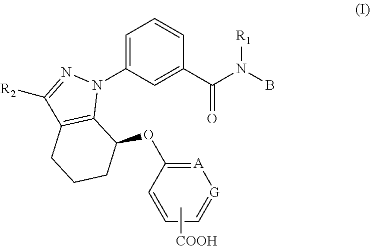

### CROSS-REFERENCE TO RELATED APPLICATIONS

This patent application claims priority from Italian patent application no. 102021000003794 filed on Feb. 18, 2021, the entire disclosure of which is incorporated herein by reference.

### TECHNICAL FIELD

The present invention relates to a method for the preparation of pyrazole derivatives useful as modulators of CFTR protein.

### BACKGROUND ART

Pyrazole derivatives are known from WO 2018/167690.

WO 2018/167690 discloses compounds of formula (Ia)

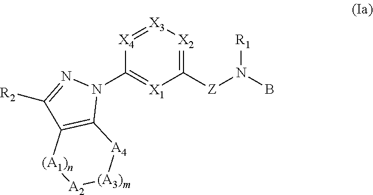

These compounds are useful as modulators of CFTR protein, in particular for the treatment of cystic fibrosis.

WO 2018/167690 discloses the following method for the preparation of compounds of formula (I):

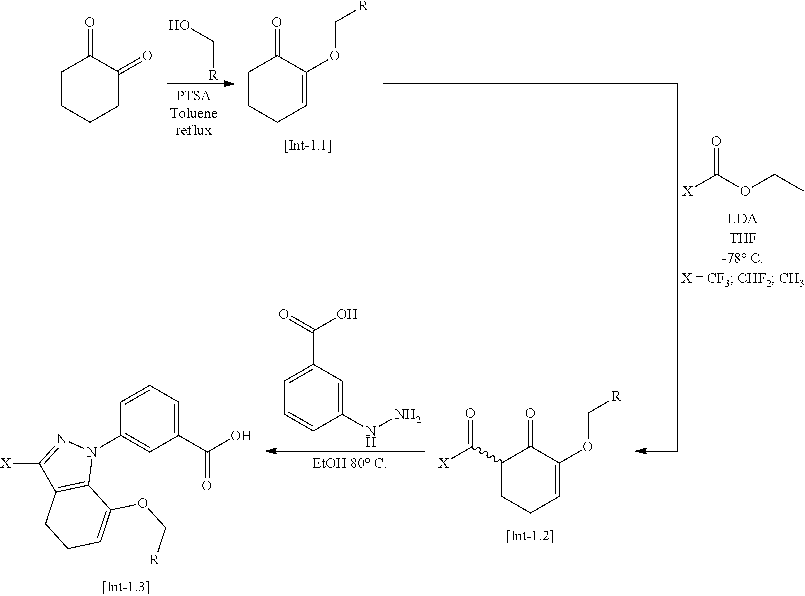

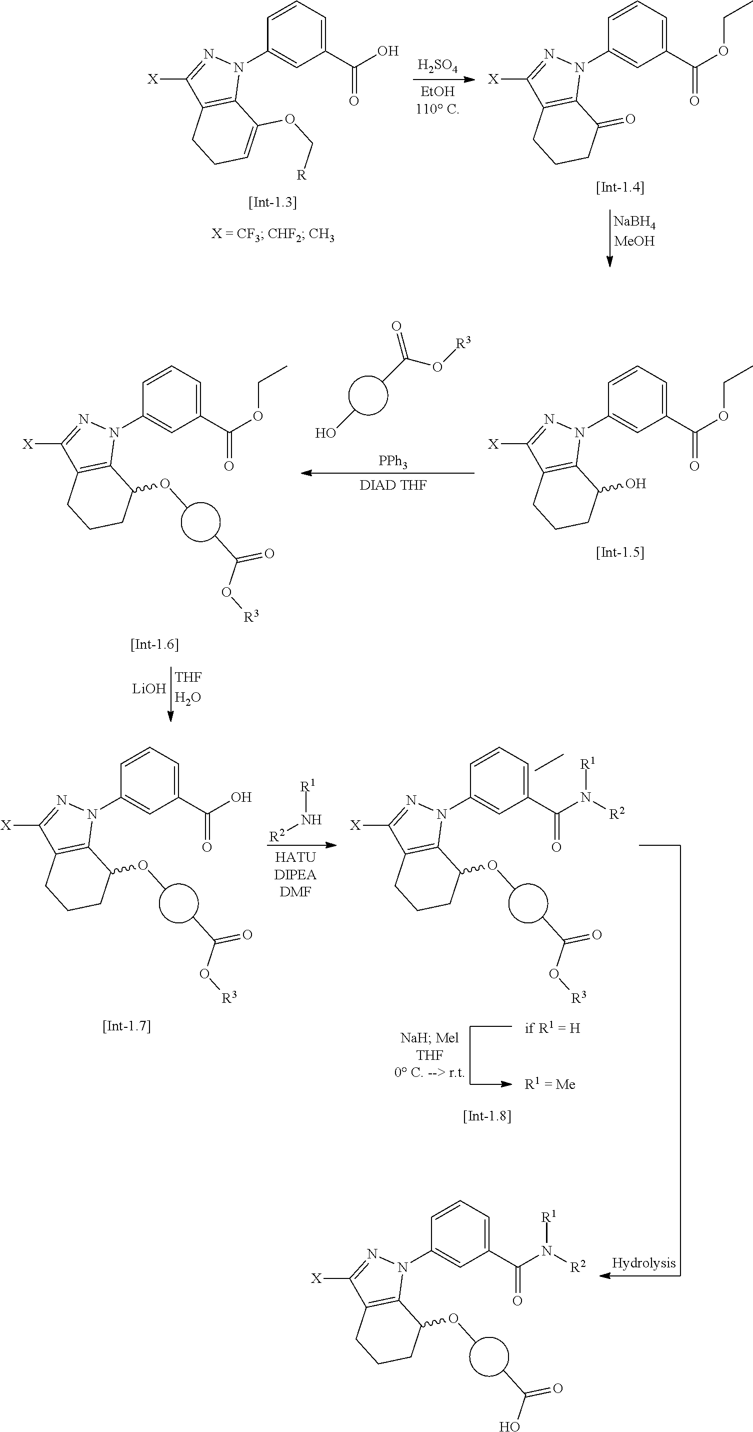

The final compounds are obtained as mixture of enantiomers, and there is the need of separating the enantiomers since they usually have different biological activity. This strongly reduces the overall yield of the process, as half of the final product is not of interest because its stereochemistry at the chiral center negatively affects the biological activity. This method is not suitable to be industrially scalable primarily due to the use of dangerous, scale-limiting reagents and reactants, requiring also the use of reaction conditions not applicable to an industrial scale. Additionally, the indicated synthetic protocol leads to the desired final compounds after a relatively long sequence of synthetic steps, in a relatively low overall yield.

In view of the above, there is a need for a new, higher-yielding, enantioselective and industrially scalable procedure for the synthesis of pyrazole derivatives useful as CFTR modulators.

### DISCLOSURE OF INVENTION

Therefore, the aim of the present invention is to provide a new method for the synthesis of pyrazole derivatives with high yield of pure product that is easy, shorter, enantioselective, and industrially scalable.

The aforementioned objective has been met according to the method of claim 1 and intermediates of claim 11. Preferred embodiments are set out within the dependent claims.

In particular, the present inventors have developed an improved process for the preparation of pure pyrazole derivatives that is industrially feasible and viable, with the use of industrially friendly reagents and solvents, and does not require cumbersome work up, chromatographic purifications, and time delaying steps.

According to a first aspect of the present invention a method for the preparation of compounds of formula (I) or pharmaceutically acceptable salts, isotopes or solvates thereof is provided

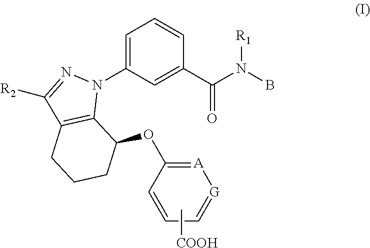

wherein:
R₁ is selected from the group consisting of C₁₋₆alkyl and C₃₋₆cycloalkyl;
R₂ is selected from the group consisting of C₁₋₆alkyl, haloC₁₋₆alkyl, C₃₋₆cycloalkyl, C₁₋₆alkyl-O—C₁₋₆alkyl, C₁₋₆alkyl-O—C₃₋₆cycloalkyl, and C₁₋₆alkyl-O-heterocycloalkyl;
B represents an unsubstituted or a substituted aromatic or heteroaromatic ring selected from the group consisting of:

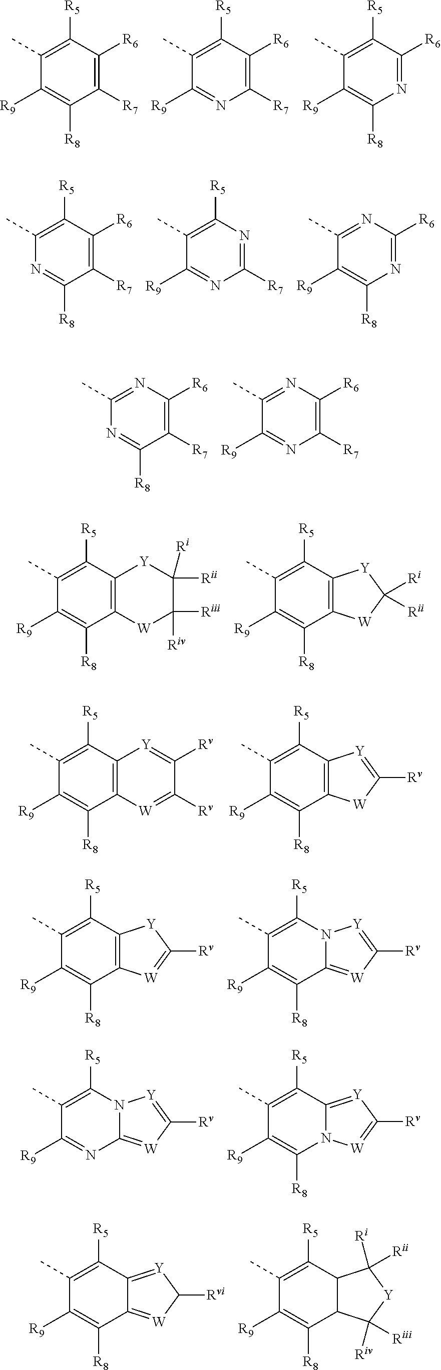

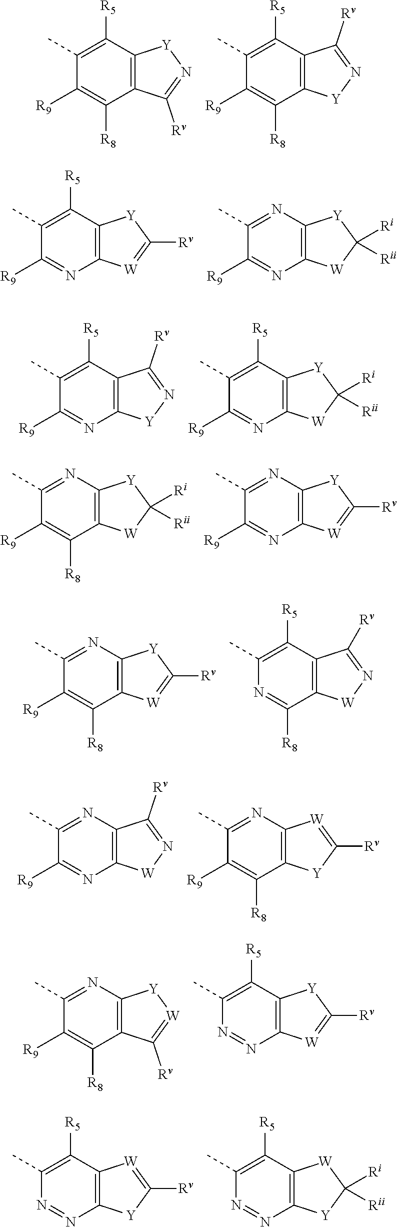

wherein
R₅, R₆, R₇, R₈, and R₉ are independently selected from the group consisting of hydrogen, halogen, C₁₋₆alkyl, haloC₁₋₆alkyl, C₃₋₆cycloalkyl, O—C₁₋₆alkyl, O—C₃₋₆cycloalkyl, O-heterocycloalkyl, O-haloC₁₋₆alkyl, COR^{viii}, COOR^{viii}, CONHR^{viii}, CONR^{viii}R^{ix}, OH, CN, NR^{x}R^{xi}, N(R^{ix})COR^{x}, N(R^{ix})CONR^{x}R^{xi} and hydroxy-C₁₋₆alkyl or
when R₆ and R₇ are present on a 6-membered heteroaromatic ring, taken together with the carbon atoms to whom they are bound, they can form a saturated or unsaturated 5-membered or 6-membered carbocyclic ring or a 5-membered or 6-membered heterocycloalkyl containing from 1 to 3 heteroatoms selected from O, N, and S or a 5-membered or 6-membered heteroaryl ring containing from 1 to 3 heteroatoms selected from O, N, and S;
Y and W are independently selected from the group consisting of O, S, SO₂, CR^{iv}R^{v}, CR^{v}, N, and NR^{vi};
Rⁱ, Rⁱⁱ, Rⁱⁱⁱ and R^{iv} are independently selected from the group consisting of hydrogen, C₁₋₆alkyl, haloC₁₋₆alkyl, halogen, OH, O—C₁₋₆alkyl and O-haloC₁₋₆alkyl or
when Rⁱ and Rⁱⁱ, or Rⁱⁱⁱ and R^{iv} are taken together with the carbon atoms to whom they are bound, they can represent C═O;
R^{v} is selected from the group consisting of hydrogen, C₁₋₆alkyl, haloC₁₋₆alkyl, O—C₁₋₆alkyl, halogen, C₃₋₆cycloalkyl, OH and O-haloC₁₋₆alkyl;
R^{vi} is selected from the group consisting of hydrogen and C₁₋₆alkyl;
R^{viii} is selected from the group consisting of hydrogen, C₁₋₆alkyl, C₃₋₆cycloalkyl, aryl, heteroaryl, heterocycloalkyl, hydroxy-C₁₋₆alkyl and C₁₋₆alkyl-O—C₁₋₆alkyl;
R^{ix} is selected from the group consisting of hydrogen, C₁₋₆alkyl, hydroxy-C₁₋₆alkyl, and C₁₋₆alkyl-O—C₁₋₆alkyl;
R^{x} is selected from the group consisting of hydrogen, C₁₋₆alkyl, C₃₋₆cycloalkyl, heterocycloalkyl, aryl, heteroaryl, hydroxy-C₁₋₆alkyl, and C₁₋₆alkyl-O—C₁₋₆alkyl;
R^{xi} is selected from the group consisting of hydrogen, C₁₋₆alkyl, hydroxy-C₁₋₆alkyl, aryl-C₁₋₆alkyl, heteroaryl-C₁₋₆alkyl, and heterocycloalkyl-C₁₋₆alkyl;
A and G are independently selected from CH and N.

Preferably A and G are both CH.

Preferably, R₅, R₆, R₇, R₈, and R₉ are independently selected from the group consisting of hydrogen, halogen, C₁₋₆alkyl, haloC₁₋₆alkyl, C³⁻⁶cycloalkyl, O—C₁₋₆alkyl, O—C₃₋₆cycloalkyl, O-heterocycloalkyl and O-haloC₁₋₆alkyl.

The method of the invention comprises the step of e), an enantioselective reduction of the ketone functionality of a compound of formula (INT-7)

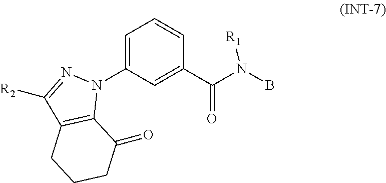

in the presence of a ruthenium-based catalyst to obtain a compound of formula (INT-8)

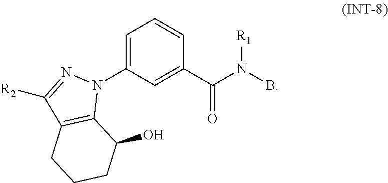

According to one embodiment, the ruthenium-based catalyst is a ruthenium complex selected from the group consisting of (S,S)-TsDPEN-RuCl(p-cymene) (i.e., [N-[(1S,2S)-2-(Amino-κN)-1,2-diphenylethyl]-4-methylbenzenesulfonamidato-κN]chloro[(1,2,3,4,5,6-η)-1-methyl-4-(1-methylethyl)benzene]-ruthenium), (S,S)-Teth-TsDpen RuCl, (S,S)-Ts-DENEB RuCl, preferably (S,S)-TsDPEN-RuCl(p-cymene).

The compound of formula INT-8 is then etherified to obtain a compound of formula (I).

Advantageously, the present invention provides an easy, high yield, cost effective, and industrially scalable process. A further advantage of the present invention is the enantioselectivity of the reduction of ketone functionality into a secondary alcohol as in step e) that allows avoiding the separation of enantiomers that was required when using the method described in WO 2018/167690.

According to a second embodiment of the invention, the method comprises, after step e), the steps of f) etherification of a compound of formula (INT-8) with a compound of formula (SM5)

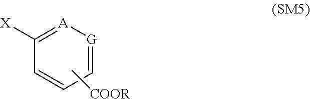

wherein R is C₁-C₄ linear or branched alkyl, preferably methyl and t-butyl, and X is halogen, preferably Cl or Br, to obtain a compound of formula (INT-9)

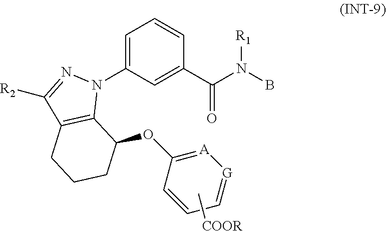

Preferably, the etherification step is carried out in the presence of a palladium catalyst and a di-1-tert-butyl-substituted bipyrazolylphosphine ligand. The palladium catalyst is preferably Pd(OAc)₂. The bipyrazolylphosphine ligand is preferably 5-(di-tert-butylphosphino)-1′,3′,5′-triphenyl-1′H-1,4′-bipyrazole (BippyPhos).

According to a third embodiment, the method further comprises, after step f), the step of g) deprotection of the ester group of compound of formula (INT-9) to obtain a compound of formula (I).

According to a fourth embodiment, the method further comprises, before step e), the step of:
d) reacting a compound of formula (INT-3)

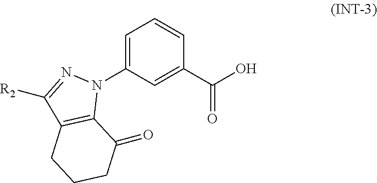

with a compound of formula (INT-6)

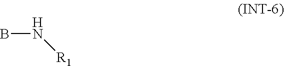

to obtain a compound of formula (INT-7).

According to a fifth embodiment, the method further comprises, before step d), the steps of:
a) selective mono protection of one of the ketone groups of a compound of formula (SM1)

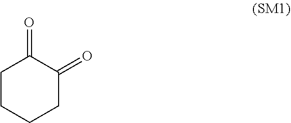

with a compound of formula HO—CH₂—Rn wherein Rn is selected from the group consisting of H, C₁-C₄ linear or branched alkyl, and phenyl to obtain a compound of formula (INT-1)

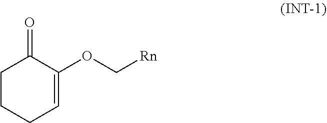

b) condensation of the compound of formula (INT-1) with a compound of formula (SM2)

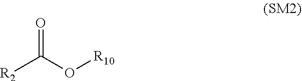

wherein R₁₀ is C₁-C₄ alkyl, to obtain a compound of formula (INT-2)

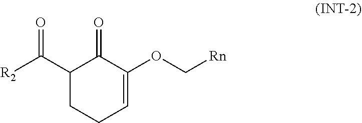

c) reacting a compound of formula (INT-2) with 3-hydrazinobenzoic acid to obtain a compound of formula (INT-3).

The method according to the invention can be used for the preparation of a compound of formula (I) selected from the group consisting of:
4-[[(S)-1-[3-[2,3-dihydrobenzofuran-6-yl(methyl)carbamoyl]phenyl]-3-(trifluoromethyl)-4,5,6,7-tetrahydroindazol-7-yl]oxy]benzoic acid;
4-[[(S)-1-[3-[methyl-(2-methyl-1,3-benzoxazol-6-yl)carbamoyl]phenyl]-3-(trifluoromethyl)-4,5,6,7-tetrahydroindazol-7-yl]oxy]benzoic acid;
4-[[(S)-1-[3-[methyl-(2-methyloxazolo[4,5-b]pyridin-6-yl)carbamoyl]phenyl]-3-(trifluoromethyl)-4,5,6,7-tetrahydroindazol-7-yl]oxy]benzoic acid;
4-[[(S)-1-[3-[[2-(difluoromethoxy)-4-pyridyl]-methyl-carbamoyl]phenyl]-3-(trifluoromethyl)-4,5,6,7-tetrahydroindazol-7-yl]oxy]benzoic acid;
4-[[1-[3-[6,7-dihydro-5H-cyclopenta[b]pyridin-3-yl(methyl)carbamoyl]phenyl]-3-(trifluoromethyl)-4,5,6,7-tetrahydroindazol-7-yl]oxy]benzoic acid;
4-[[(S)-1-[3-[2,3-dihydrofuro[3,2-b]pyridin-6-yl(methyl)carbamoyl]phenyl]-3-(trifluoromethyl)-4,5,6,7-tetrahydroindazol-7-yl]oxy]benzoic acid;
3-[[(S)-1-[3-[(2,2-difluoro-1,3-benzodioxol-5-yl)-methyl-carbamoyl]phenyl]-3-(trifluoromethyl)-4,5,6,7-tetrahydroindazol-7-yl]oxy]benzoic acid;
4-[[1-[3-[(2,2-difluoro-1,3-benzodioxol-5-yl)-methyl-carbamoyl]phenyl]-3-(difluoromethyl)-4,5,6,7-tetrahydroindazol-7-yl]oxy]benzoic acid;
6-[[(S)-1-[3-[(2,2-difluoro-1,3-benzodioxol-5-yl)-methyl-carbamoyl]phenyl]-3-(trifluoromethyl)-4,5,6,7-tetrahydroindazol-7-yl]oxy]pyridine-3-carboxylic acid;
5-[[(S)-1-[3-[(2,2-difluoro-1,3-benzodioxol-5-yl)-methyl-carbamoyl]phenyl]-3-(trifluoromethyl)-4,5,6,7-tetrahydroindazol-7-yl]oxy]pyridine-3-carboxylic acid.

Preferably, the compound of formula (I) is (S)-4-[[1-[3-[(2,2-difluoro-1,3-benzodioxol-5-yl)-methyl-carbamoyl]phenyl]-3-(trifluoromethyl)-4,5,6,7-tetrahydroindazol-7-yl]oxy] benzoic acid (ARN23765).

According to a further aspect of the invention, the following compounds are also provided as reaction intermediates selected from the group consisting of:
N,2-dimethyloxazolo[4,5-b]pyridin-6-amine;
2-(difluoromethoxy)-N-methyl-pyridin-4-amine;
N-methyl-6,7-dihydro-5H-cyclopenta[b]pyridin-3-amine;
-methyl-2,3-dihydrofuro[3,2-b]pyridin-6-amine
N-(2,3-dihydrobenzofuran-6-yl)-N-methyl-3-[7-oxo-3-(trifluoromethyl)-5,6-dihydro-4H-indazol-1-yl]benzamide;
N-methyl-N-(2-methyl-1,3-benzoxazol-6-yl)-3-[7-oxo-3-(trifluoromethyl)-5,6-dihydro-4H-indazol-1-yl]benzamide;
N-methyl-N-(2-methyloxazolo[4,5-b]pyridin-6-yl)-3-[7-oxo-3-(trifluoromethyl)-5,6-dihydro-4H-indazol-1-yl]benzamide;
N-[2-(difluoromethoxy)-4-pyridyl]-N-methyl-3-[7-oxo-3-(trifluoromethyl)-5,6-dihydro-4H-indazol-1-yl]benzamide;
N-(6,7-dihydro-5H-cyclopenta[b]pyridin-3-yl)-N-methyl-3-[7-oxo-3-(trifluoromethyl)-5,6-dihydro-4H-indazol-1-yl]benzamide;
N-(2,3-dihydrofuro[3,2-b]pyridin-6-yl)-N-methyl-3-[7-oxo-3-(trifluoromethyl)-5,6-dihydro-4H-indazol-1-yl]benzamide;
N-(2,2-difluoro-1,3-benzodioxol-5-yl)-3-[3-(difluoromethyl)-7-oxo-5,6-dihydro-4H-indazol-1-yl]-N-methyl-benzamide.

### BEST MODE FOR CARRYING OUT THE INVENTION

In the following, the present invention shall be illustrated by means of some examples, which are not construed to be viewed as limiting the scope of the invention.

### Example

Compound ARN23765 has been prepared using the synthetic pathway according to Scheme 1.

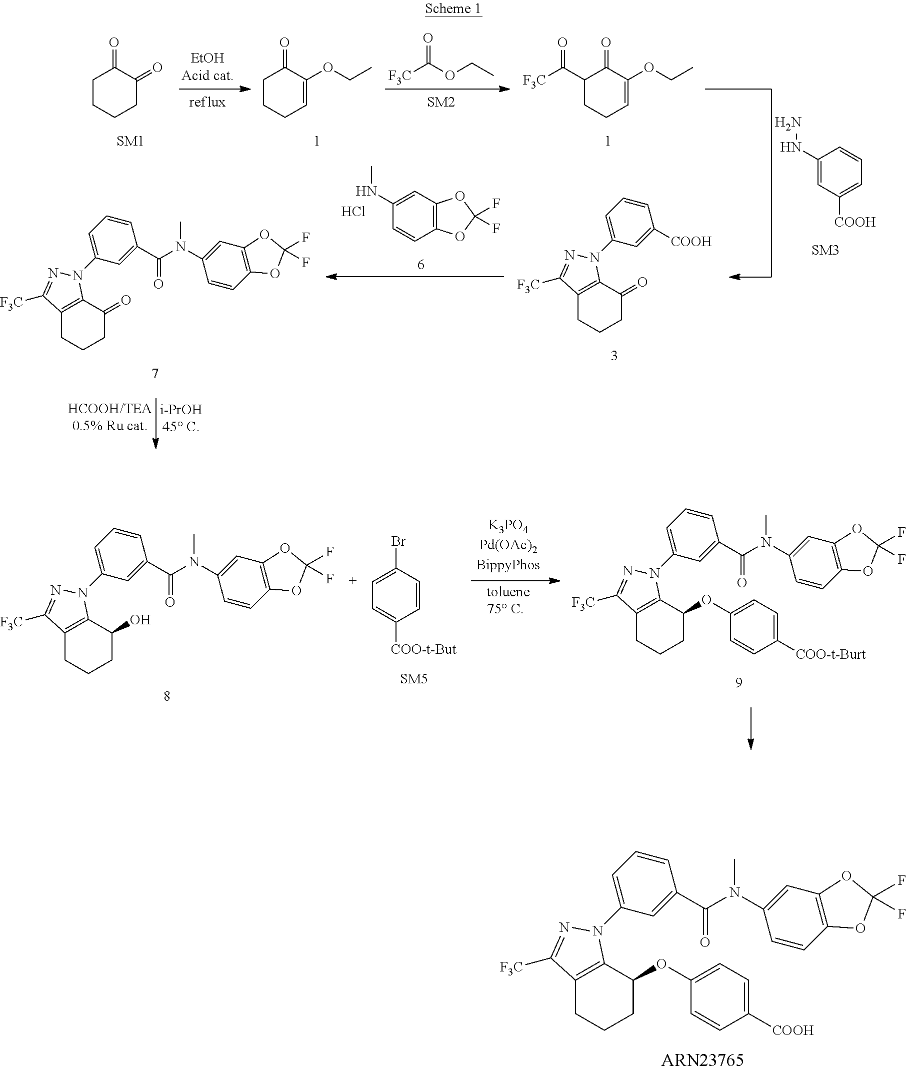

### Stage 1: Preparation of Compound 3

In a 10 L reactor, SM1 (300 g, 1 eq) was dissolved in 5 volumes (vol) of EtOH. H₃PO₄ (0.5 eq) was dissolved in EtOH (5 vol) and added in 10 minutes. The reaction mixture was heated to reflux and volumes reduced to 3 by distillation. Then, 5 volumes of fresh EtOH were added and volumes reduced to 3. This operation was performed few times, until HPLC control showed residual amount of SM1. The reaction was left overnight at reflux in 10 total volumes, then monitored again to show circa 20% a/a of SM1. Few more operations of distilling and charging of EtOH were performed until HPLC indicated full consumption of SM1. The reaction volumes were then restored to 10 by adding fresh EtOH, and temperature lowered to 20° C. Upon full conversion of SM1, the reaction quenching was performed by adding 1 eq of solid K₃PO₄ to the reaction mixture, then 4 vol of water were charged in 30 minutes. Once the solid was completely dissolved, two clear phases were obtained. The bottom phase (water) was discarded. Volumes were reduced to 3 by distillation at atmospheric pressure, then 5 Vol of THF were added and distilled four times in total.

The temperature was lowered to 0° C., which induced phosphate salts precipitation. The resulting solution was filtered and washed with 4 vol of THF.

The reaction volumes were then reduced to 2 by distillation at atmospheric pressure, the temperature was lowered to −10° C., and SM2 (1.5 eq) was charged. LiHMDS 1M in THF (1.3 eq, 11.13 vol) was added dropwise in 2 h. At the end of the addition, upon full consumption of compound 1, the reaction was considered completed. The temperature was set to 0° C. and the reaction quenched by adding 3 eq of TFA in 1 h and stirring the solution for 30 min. The temperature was then set to 15° C., SM3 (0.85 eq) was divided into 4 portions and added every 15 minutes. After the last addition, temperature was raised to 20° C. in 60 min. The reaction mixture was left under stirring overnight at 20° C.

To reach full conversion of enol-ether 3, the temperature was increased and the reaction was refluxed for 2 h. The reaction was filtered through silica pad overnight. At the end of the filtration, the reaction volumes were reduced to a total of 8 by vacuum distillation.

Methyl-tert-butyl ether (5 vol) and water (10 vol) were added, followed by dropwise addition of 3 vol of NaOH 35% w/w over 1 h. The temperature was set to 20° C. and the solution allowed to separate. The upper phase was discarded and the aqueous phase was washed a second time with 5 vol of MTBE, the solution mixed for 15 min and then allowed to separate. The resulting aqueous phase was then charged into a reactor with 2 vol of MTBE, then the temperature was set to 0° C. and 2.5 vol of H₃PO₄ were added dropwise in 30 minutes. The temperature was set to 20° C., the mixture stirred for 15 minutes and the bottom water phase was discarded. Then, 2.5 L of EtOH were added to the organic phase and the solution left overnight at 20° C.

The following day, the reaction volumes were reduced to 5 vol total by evaporation at atmospheric pressure, then 10 vol of water were added in 1 h, maintaining the internal temperature above 70° C. The external temperature was decreased from 90° C. to 20° C. in 180 minutes. A product started to appear as a creamy slurry with beige color. The desired compound was filtered on a P3 sintered funnel, and the cake washed with 10 volumes of a 2:8 EtOH/water solution.

The product was deliquored for 8 hours, then dried under vacuum at 40° C. for 48 h to afford 569 g of compound 3.

### Stage 2: Preparation of Compound 6

Compound 6 is prepared according to the synthetic preparation disclosed in WO 2018/167690.

### Stage 3: Preparation of Compound 7

In a 5 L reactor, compound 3 (555 g) and compound 6 (421 g, 1 eq) were suspended in AcOEt (4 vol) to give a slurry. Triethyl amine (TEA, 2 vol) was added and the mixture heated to 40° C. A propylphosphonic anhydride (T3P) solution (2.2 vol) was added dropwise in 30 min and, at the end of the addition, the suspension was heated to reflux for 5 hours. The temperature was decreased to 20° C. over 3 hours and stirred for 8 hours.

The temperature was set to 30° C., water (1 vol) was added, and layers were allowed to separate. The organic phase was concentrated to 1.5 volumes at ambient pressure, then MTBE (4 vol) was added and volumes reduced to 2 at ambient pressure. Additional 4 vol of MTBE were added and volumes reduced to 3 total (1.6 L). The temperature was lowered to 20° C. in 3 hours and the reaction mixture was left stirring for 10 hours at the same temperature, giving a solid precipitate. Then, 2 vol of n-heptane were added, which turned the slurry into a gummy solid. T_{jack} was set to 50° C. and 5 vol of MTBE were added. Volumes were reduced under vacuum to 2 and this operation was repeated a second time. After this step, the temperature was set to 20° C. and the slurry was left stirring for 4 hours. The solid was filtered on a P3 sintered funnel, washed with 3 vol of n-heptane and de-liquored for 1 hour to give 700 g of compound 7. Titer determination with hydroquinone indicated a purity of 80% w/w.

### Stage 4: Preparation of Compound 9

In a 20 L reactor, compound 7 (700 g, 1 eq), was suspended in isopropyl alcohol (IPA) (10 vol) at room temperature, trimethylamine (TEA, 0.6 vol) and the catalyst (S,S)-TsDPENRuCl(p-cymene) (0.006 wt) were added to the mixture, and 3 vacuum/nitrogen cycles were applied. Formic acid (0.4 vol) was added slowly to the mixture in 10 minutes. The internal temperature was increased to 45° C. and the solution stirred until HPLC control indicated full consumption of compound 7. The reaction mixture changed from an initial slurry to a solution upon reaction completion. The reaction mixture was concentrated under vacuum at 55° C. to residual 2 vol, then the solvent was swapped with ethyl acetate (AcOEt, 10 vol) and concentrated to 2 vol. To remove residual amount of IPA, AcOEt (4 vol) and water (1 vol) were added and, after stirring, layers were separated. Acetyl cysteine (1 vol, 10% w/w solution) was added and the bi-phasic solution stirred for 3 hours. Phases were separated and the organic phase was passed through a silica pad (0.5 wt) and washed with AcOEt (4 vol). The organic layer was treated with 5% NaHCO₃ aqueous solution (1.2 vol), then charged in the reactor and concentrated to 2 vol under vacuum. Toluene (10 vol) was charged and concentrated to 2 vol at 55° C. under vacuum; ¹H-NMR assay with hydroquinone indicated a total of 600 g of compound 8 obtained. In a second 5 L reactor, K₃PO₄ (789 g, 4 eq based on theoretical 527 g of compound 8) and toluene (10 vol) were charged and concentrated to 2 vol at atmospheric pressure, then temperature was lowered to 55° C. The toluene solution of 527 g of compound 8 (2 vol) was charged in the 5 L reactor, then SM5 (0.63 wt) was added. The resulting mixture was concentrated to 3 vol, then BippyPhos (0.02 wt) and Pd(OAc)₂ (0.0046 wt) were added. Three vacuum/nitrogen cycles were applied and the temperature was set to 75° C.; the reaction was completed in 44 hours. The resulting mixture was cooled to 40° C., then water (2 vol) was added. After stirring for 20 min, the layers were separated. The temperature was set to 50° C., NaHSO₃ (2 vol, 5% w/w solution) was added, and the biphasic solution stirred for 16 hours. Phases were separated and the organic layer passed through Sterimat® and washed with toluene (1 vol). The filtrate was charged in the reactor and concentrated under vacuum to 2 vol. Then, IPA (5 vol) was added and the mixture concentrated at 55° C. under vacuum to 3 vol; additional IPA (5 vol) was charged and concentrated at atmospheric pressure to 2 vol, n-Heptane (7 vol) was charged and the mixture stirred for 1 hour at 50° C., then cooled to 20° C. in 1 hour and aged overnight. The solid was filtered and washed with 3 vol n-heptane. The solid was dried under vacuum at 40° C. to give 590 g of compound 9.

### Stage 5: Preparation of ARN23765

In a 5 L reactor, compound 9 was charged (581 g) and dissolved in acetonitrile (5 vol), then HCl 37% w/w was added (0.25 vol). After 23 h (18+5), the reaction was concentrated.

The mixture was concentrated to 2 vol at 55° C. under vacuum, then IPA (5 vol) was added and concentrated to final 3 vol at 55° C. under vacuum. Finally, IPA (5 vol) was added and concentrated to final 3 vol at atmospheric pressure. n-Heptane (1 vol) was added and the suspension cooled to 20° C. in 1 hour and aged overnight. The suspension was filtered on P3 glass filter and washed with IPA/n-heptane (0.5/2.5 vol) to give the final ARN23765 with a HPLC purity >99.9% a/a. The solid was dried in oven at 45° C. under vacuum overnight to give 374.5 g of white solid.

1. Method for the preparation of a compound of formula (I) 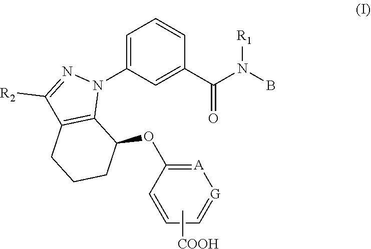
or pharmaceutically acceptable salts, isotopes or solvates thereof wherein:
R₁ is selected from the group consisting of C₁₋₆alkyl and C₃₋₆cycloalkyl;
R₂ is selected from the group consisting of C₁₋₆alkyl, haloC₁₋₆alkyl, C₃₋₆cycloalkyl, C₁₋₆alkyl-O—C₁₋₆alkyl, C₁₋₆alkyl-O—C₃₋₆cycloalkyl and C₁₋₆alkyl-O-heterocycloalkyl;
B represents an unsubstituted or a substituted aromatic or heteroaromatic ring selected from the group consisting of: 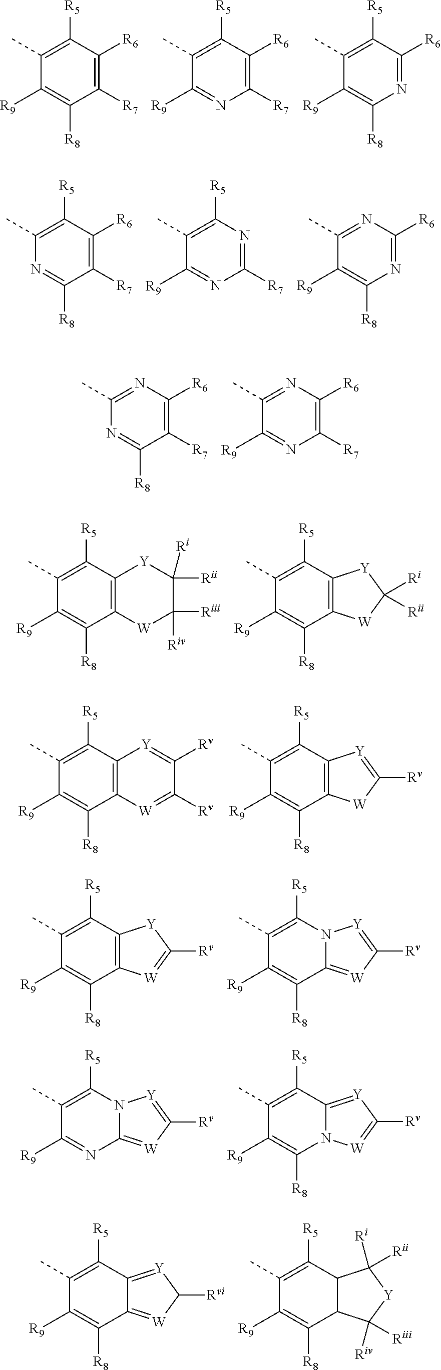 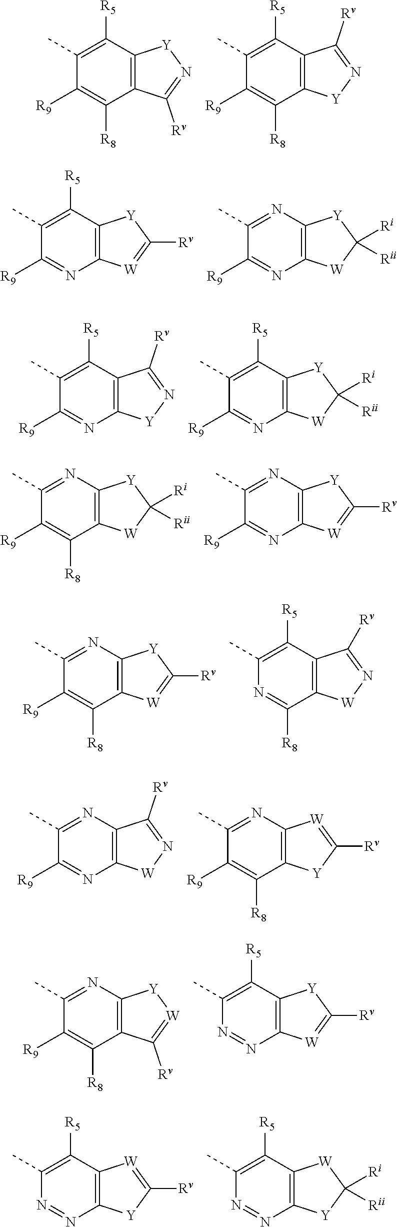
wherein
R₅, R₆, R₇, R₈, and R₉ are independently selected from the group consisting of hydrogen, halogen, C₁₋₆alkyl, haloC₁₋₆alkyl, C₃₋₆cycloalkyl, O—C₁₋₆alkyl, O—C₃₋₆cycloalkyl, O-heterocycloalkyl, O-haloC₁₋₆alkyl, COR^{viii}, COOR^{viii}, CONHR^{viii}, CONR^{viii}R^{ix}, OH, CN, NR^{x}R^{xi}, N(R^{ix})COR^{x}, N(R^{ix})CONR^{x}R^{xi} and hydroxy-C₁₋₆alkyl, or
when R₆ and R₇ are present on a 6-membered heteroaromatic ring, taken together with the carbon atoms to whom they are bound, they can form a saturated or unsaturated 5-membered or 6-membered carbocyclic ring or a 5-membered or 6-membered heterocycloalkyl containing from 1 to 3 heteroatoms selected from O, N, and S or a 5-membered or 6-membered heteroaryl ring containing from 1 to 3 heteroatoms selected from O, N, and S;
Y and W are independently selected from the group consisting of O, S, SO₂, CR^{iv}R^{v}, CR^{v}, N, and NR^{vi};
Rⁱ, Rⁱⁱ, Rⁱⁱⁱ and R^{iv} are independently selected from the group consisting of hydrogen, C₁₋₆alkyl, haloC₁₋₆alkyl, halogen, OH, O—C₁₋₆alkyl and O-haloC₁₋₆alkyl or
when Rⁱ and Rⁱⁱ, or Rⁱⁱⁱ and R^{iv} are taken together with the carbon atoms to whom they are bound, they can represent C═O;
R^{v} is selected from the group consisting of hydrogen, C₁₋₆alkyl, haloC₁₋₆alkyl, O—C₁₋₆alkyl, halogen, C₃₋₆cycloalkyl, OH and O-haloC₁₋₆alkyl;
R^{vi} is selected from the group consisting of hydrogen and C₁₋₆alkyl;
R^{viii} is selected from the group consisting of hydrogen, C₁₋₆alkyl, C₃₋₆cycloalkyl, aryl, heteroaryl, heterocycloalkyl, hydroxy-C₁₋₆alkyl and C₁₋₆alkyl-O—C₁₋₆alkyl;
R^{ix} is selected from the group consisting of hydrogen, C₁₋₆alkyl, hydroxy-C₁₋₆alkyl, and C₁₋₆alkyl-O—C₁₋₆alkyl;
R^{x} is selected from the group consisting of hydrogen, C₁₋₆alkyl, C₃₋₆cycloalkyl, heterocycloalkyl, aryl, heteroaryl, hydroxy-C₁₋₆ alkyl, and C₁₋₆alkyl-O—C₁₋₆alkyl;
R^{xi} is selected from the group consisting of hydrogen, C₁₋₆alkyl, hydroxy-C₁₋₆alkyl, C₁₋₆alkyl-O—C₁₋₆alkyl, aryl-C₁₋₆alkyl, heteroaryl-C₁₋₆alkyl, and heterocycloalkyl-C₁₋₆alkyl;
A and G are independently selected from CH and N;
the method comprising a step of e) enantioselective reduction of the ketone functionality of a compound of formula (INT-7) 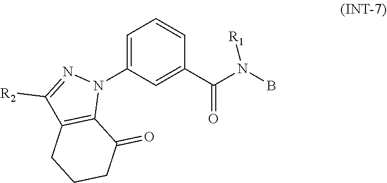
in the presence of a ruthenium-based catalyst to obtain a compound of formula (INT-8)

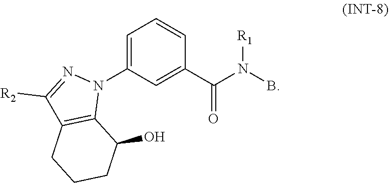

2. The method according to claim 1 wherein the ruthenium-based catalyst is a ruthenium complex selected from the group consisting of (S,S)-TsDPEN-RuCl(p-cymene), (S,S)-Teth-TsDpen RuCl, and (S,S)-Ts-DENEB RuCl.

3. The method according to claim 2, wherein the ruthenium-based catalyst is (S,S)-TsDPEN-RuCl(p-cymene).

4. The method according to claim 1 further comprising, after step e), the step of f) etherification of a compound of formula (INT-8) with a compound of formula (SM5) 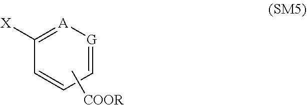
wherein R is a C₁-C₄ linear or branched alkyl and X is halogen, to obtain a compound of formula (INT-9)

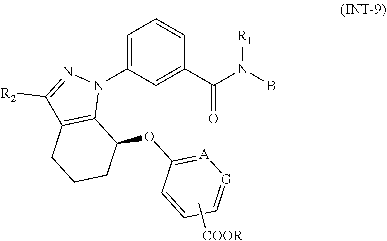

5. The method according to claim 4, wherein the etherification step is carried out in the presence of a palladium catalyst and a di-1-tert-butyl-substituted bipyrazolylphosphine ligand.

6. The method according to claim 4, further comprising the step of g) deprotection the ester group of a compound of formula (INT-9) to obtain a compound of formula (I).

7. The method according to claim 1 characterized in that it further comprises, before step e), the step of:
d) reacting a compound of formula (INT-3) 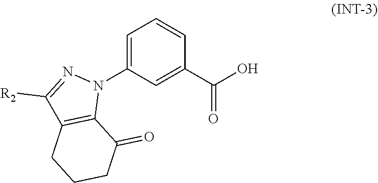
with a compound of formula (INT-6) 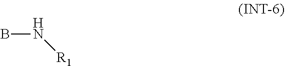
to obtain a compound of formula (INT-7).

8. The method according to claim 7, characterized in that it further comprises, before step d), the steps of:
a) selective mono protection of one of the ketone groups of a compound of formula (SM1) 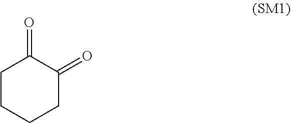
with a compound of formula HO—CH₂—Rn wherein Rn is selected from the group consisting of H, C₁-C₄ linear or branched alkyl, and phenyl to obtain a compound of formula (INT-1) 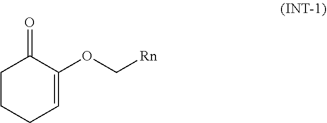
b) condensation of the compound of formula (INT-1) with a compound of formula (SM2) 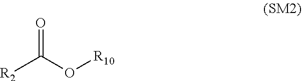
wherein R₁₀ is C₁-C₄ alkyl, to obtain a compound of formula (INT-2) 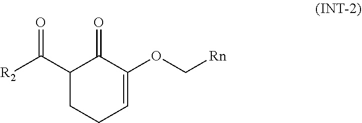
c) reacting a compound of formula (INT-2) with 3-hydrazinobenzoic acid to obtain a compound of formula (INT-3).

9. The method according to claim 1 wherein the compound of formula (I) is selected from the group consisting of:
4-[[(S)-1-[3-[2,3-dihydrobenzofuran-6-yl(methyl)carbamoyl]phenyl]-3-(trifluoromethyl)-4,5,6,7-tetrahydroindazol-7-yl]oxy]benzoic acid;
4-[[(S)-1-[3-[methyl-(2-methyl-1,3-benzoxazol-6-yl)carbamoyl]phenyl]-3-(trifluoromethyl)-4,5,6,7-tetrahydroindazol-7-yl]oxy]benzoic acid;
4-[[(S)-1-[3-[methyl-(2-methyloxazolo[4,5-b]pyridin-6-yl)carbamoyl]phenyl]-3-(trifluoromethyl)-4,5,6,7-tetrahydroindazol-7-yl]oxy]benzoic acid;
4-[[(S)-1-[3-[[2-(difluoromethoxy)-4-pyridyl]-methyl-carbamoyl]phenyl]-3-(trifluoromethyl)-4,5,6,7-tetrahydroindazol-7-yl]oxy]benzoic acid;
4-[[1-[3-[6,7-dihydro-5H-cyclopenta[b]pyridin-3-yl(methyl)carbamoyl]phenyl]-3-(trifluoromethyl)-4,5,6,7-tetrahydroindazol-7-yl]oxy]benzoic acid;
4-[[(S)-1-[3-[2,3-dihydrofuro[3,2-b]pyridin-6-yl(methyl)carbamoyl]phenyl]-3-(trifluoromethyl)-4,5,6,7-tetrahydroindazol-7-yl]oxy]benzoic acid;
3-[[(S)-1-[3-[(2,2-difluoro-1,3-benzodioxol-5-yl)-methyl-carbamoyl]phenyl]-3-(trifluoromethyl)-4,5,6,7-tetrahydroindazol-7-yl]oxy]benzoic acid;
4-[[1-[3-[(2,2-difluoro-1,3-benzodioxol-5-yl)-methyl-carbamoyl]phenyl]-3-(difluoromethyl)-4,5,6,7-tetrahydroindazol-7-yl]oxy]benzoic acid;
6-[[(S)-1-[3-[(2,2-difluoro-1,3-benzodioxol-5-yl)-methyl-carbamoyl]phenyl]-3-(trifluoromethyl)-4,5,6,7-tetrahydroindazol-7-yl]oxy]pyridine-3-carboxylic acid; and
5-[[(S)-1-[3-[(2,2-difluoro-1,3-benzodioxol-5-yl)-methyl-carbamoyl]phenyl]-3-(trifluoromethyl)-4,5,6,7-tetrahydroindazol-7-yl]oxy]pyridine-3-carboxylic acid.

10. The method according to claim 1 wherein the compound of formula (I) is (S)-4-[[1-[3-[(2,2-difluoro-1,3-benzodioxol-5-yl)-methyl-carbamoyl]phenyl]-3-(trifluoromethyl)-4,5,6,7-tetrahydroindazol-7-yl]oxy] benzoic acid.

11. A reaction intermediate selected from the group consisting of:
N,2-dimethyloxazolo[4,5-b]pyridin-6-amine;
2-(difluoromethoxy)-N-methyl-pyridin-4-amine;
N-methyl-6,7-dihydro-5H-cyclopenta[b]pyridin-3-amine;
N-methyl-2,3-dihydrofuro[3,2-b]pyridin-6-amine
N-(2,3-dihydrobenzofuran-6-yl)-N-methyl-3-[7-oxo-3-(trifluoromethyl)-5,6-dihydro-4H-indazol-1-yl]benzamide;
N-methyl-N-(2-methyl-1,3-benzoxazol-6-yl)-3-[7-oxo-3-(trifluoromethyl)-5,6-dihydro-4H-indazol-1-yl]benzamide;
N-methyl-N-(2-methyloxazolo[4,5-b]pyridin-6-yl)-3-[7-oxo-3-(trifluoromethyl)-5,6-dihydro-4H-indazol-1-yl]benzamide;
N-[2-(difluoromethoxy)-4-pyridyl]-N-methyl-3-[7-oxo-3-(trifluoromethyl)-5,6-dihydro-4H-indazol-1-yl]benzamide;
N-(6,7-dihydro-5H-cyclopenta[b]pyridin-3-yl)-N-methyl-3-[7-oxo-3-(trifluoromethyl)-5,6-dihydro-4H-indazol-1-yl]benzamide;
N-(2,3-dihydrofuro[3,2-b]pyridin-6-yl)-N-methyl-3-[7-oxo-3-(trifluoromethyl)-5,6-dihydro-4H-indazol-1-yl]benzamide; and
N-(2,2-difluoro-1,3-benzodioxol-5-yl)-3-[3-(difluoromethyl)-7-oxo-5,6-dihydro-4H-indazol-1-yl]-N-methyl-benzamide.
